(19) United States
(12) Patent Application Publication
(10) Pub. No.: US 2016/0106774 A1
(43) Pub. Date: Apr. 21, 2016

(54) LIQUID PHARMACEUTICAL COMPOSITION CONTAINING PIROXICAM AND HYALURONIC ACID FOR THE TREATMENT OF OSTEOARTHRITIS

(71) Applicant: DONG-A ST CO., LTD, Dongdaemun-gu, Seoul (KR)

(72) Inventors: Soon-Hoe KIM, Suwon-si (KR); Mi-Won SON, Yongin-si (KR); Sun-Woo JANG, Seoul (KR); Chan-Woong PARK, Yongin-si (KR); Kyung-Wan MA, Yongin-si (KR); Sang-Won JUNG, Yongin-si (KR)

(73) Assignee: DONG-A ST CO., LTD., Seoul (KR)

(21) Appl. No.: 14/897,571

(22) PCT Filed: May 27, 2014
(86) PCT No.: PCT/KR2014/004745

§ 371 Date: Dec. 10, 2015

(30) Foreign Application Priority Data
Jun. 13, 2013 (KR) 10-2013-0067792

(51) Int. Cl. A61K 31/728; A61K 9/00; A61K 31/5415

(52) U.S. Cl. CPC A61K 31/728; A61K 31/5415; A61K 9/0019

(57) ABSTRACT

A pharmaceutical composition for the treatment of osteoarthritis comprising piroxicam or pharmaceutically acceptable salt thereof and hyaluronic acid or pharmaceutically acceptable salt thereof at a specific ratio is provided. The composition generates synergistic effect on both anti-inflammatory and analgesic effects simultaneously. The pharmaceutical composition contains 0.25-10.0 wt % of piroxicam or its pharmaceutically acceptable salt and 0.5-5.0 wt % of hyaluronic acid or its pharmaceutically acceptable salt, wherein the weight ratio between piroxicam or its pharmaceutically acceptable salt and hyaluronic acid or its pharmaceutically acceptable salt is between 1:1 and 1:3.

### TECHNICAL FIELD

The present invention relates to a pharmaceutical composition for the treatment of osteoarthritis (degenerative arthritis) comprising piroxicam and hyaluronic acid at a specific ratio that generates synergistic effect on both anti-inflammatory and analgesic effects simultaneously.

### BACKGROUND ART

Osteoarthritis, also known as degenerative arthritis, is a disease that causes symptoms such as severe pain, joint movement disorders and others because of structural changes in joints and damages to the articular cartilage (Di Domenica et al, 2005). It is difficult to make a clear definition of osteoarthritis, because its physiological state including the level of cartilage loss and alteration in bones is assessed by radiography (Ayral et al, 2005), but it is generally defined as “a condition characterized by cartilage loss and structural change in bursa, joints, and bones around joints.” (Altman 1987; Altman et al, 1990). Assessment of knee joints of actual patients with regenerative arthritis showed that they have injury or damage to the cartilage protecting the joints or ligaments and bones that make up the joints, causing irreversible destruction of cartilage tissue. The irreversible destruction of cartilage tissue causes loss of joint mobility, impeding daily activities and causing inflammation and pain. Particularly, knee osteoarthritis is important to health of the elderly population, as it leads to pain and the physical dysfunction in the daily activities such as climbing stairs, standing up from sitting position, and walking. Moreover, it causes clinical symptoms such as impairment of joint movement, pain, muscle weakness, deformity of joint bending, varus deformity, and contracture, as well as physical impairment that significantly affects the quality of life (Kim et al, 2011).

The current treatment goal in treatment of osteoarthritis is to avoid side effects of the treatment while inhibiting the pain, improving the function of joints and the quality of life. The basic treatments of osteoarthritis include pharmacological and non-pharmacological treatments. Non-pharmacological treatments include weight loss, lower limb muscle strengthening exercises, and physical therapy. Pharmacological treatments include drug therapies such as oral administration of acetaminophen or non-steroidal anti-inflammatory drugs (NSAIDs) and intra-articular injection of steroids or sodium hyaluronate, as well as surgical methods like replacement arthroplasty (Diracoglu et al, 2009; Iwamono et al, 2007; Tunay et al, 2010).

Polymeric hyaluronic acid or a pharmaceutically acceptable salt thereof is used for treatment of degenerative arthritis or rheumatoid arthritis in the form of liquid injection. It is inserted directly into the affected parts, such as the knee and shoulder joints. It is reported that the viscoelastic polymeric substance is directly injected into the articular cavity to relieve the shock felt upon joint movement due to the loss of cartilage tissue, as well as to facilitate lubrication, thus alleviating joint pain and normalizing functions, as well as improving arthritis-caused dysfunctions and inhibiting pain (JeungTakSuh, Clinical importance and application of hyaluronic acid. Korean Journal of Family Medicine 2002; 23(9): 1071-1079; Dong Chul Lee, Seung Hee Back, Wook Jin Sohn et al. Effect of the hyaluronic acid on osteoarthritis of the knee. Journal of Korean Knee Society 2002; 14(2): 213-221; Yeong Wook Song. Pharmacological therapy in osteoarthritis. Journal of Korean Medical Association 2003; 46(11): 958-964; Seung SookNo, Jae Jun Lee, SungMi Hwang et al. Efficacy of intra-articular sodium hyaluronate in patients with osteoarthritis of the knee. The Korean Journal of Pain 2004; 17(2): 170-174).

Piroxicam, a non-steroidal anti-inflammatory drug of the benzothiazine derivative class, inhibits prostaglandin synthesis, producing anti-inflammatory effect. It is currently used to treat degenerative arthritis for its outstanding analgesic and anti-inflammatory effect as well as the long plasma half-life. It is currently used as oral medications, intramuscular injections, and patches or gels for external use.

Japanese Patent Application No. 1992-18022 discloses a stable piroxicam composition consisting of piroxicam, a water-soluble polymer, hyaluronic acid and surfactant. The composition comprises 0.1-0.5 w/v % of piroxicam and 0.03˜0.05 w/v % of hyaluronic acid. However, hyaluronic acid, water-soluble polymer and surfactant are used as an excipient to stabilize piroxicam. The hyaluronic acid is not an active ingredient in this composition.

U.S. Pat. No. 5,095,037 administered to an animal arthritis model a combination of sodium hyaluronate and non-steroidal anti-inflammatory drugs including piroxicam to assess their additive or synergistic anti-inflammatory effects. The results showed that the coadministration of indomethacin, diclofenac, or ibuprofen with sodium hyaluronate produced additive or synergistic effects in a carrageenan-induced animal podedema model, and the coadministration effect of piroxicam 4 mg/kg and sodium hyaluronate 4 mg/kg was weak. Moreover, the part inflamed by carrageenan was the foot pad, which is significantly different from body parts that are affected by actual osteoarthritis, such as knee joint, distal finger, carpometacarpus, proximal joint, metatarsal interphalangeal joint, wrist, coxa, lumbar spine, cervical spine, and ankle. Furthermore, the mechanism also differs from actual osteoarthritis. Thus, this is not suitable for evaluating the therapeutic effect of co-administration of NSAID and hyaluronic acid on osteoarthritis

As seen above, it could not be anticipated from prior art, with regard to the anti-inflammatory action and analgesic action of the composition comprising piroxicam and hyaluronic acid, that such a composition produces additive or synergistic effect on treatment of osteoarthritis.

Yet, the present inventor(s) combined piroxicam or its pharmaceutically acceptable salt and hyaluronic acid or its pharmaceutically acceptable salt at a specific ratio, and discovered that the combination produced synergistic effect simultaneously on the anti-inflammatory action and analgesic action, and thus is highly effective in treating osteoarthritis. The present inventor(s) conducted various experiments to complete the present invention.

### DISCLOSURE OF INVENTION

### Technical Problem

The object of the present invention is to provide a pharmaceutical composition for the treatment of osteoarthritis, wherein the composition comprises piroxicam or its pharmaceutically acceptable salt and hyaluronic acid or its pharmaceutically acceptable salt at a particular ratio that produces synergistic effect on both anti-inflammatory effect and analgesic effect simultaneously.

### Solution to Problem

To achieve the above object, the present invention provides a pharmaceutical composition for the treatment of osteoarthritis, comprising piroxicam or its pharmaceutically acceptable salt at 0.25-10.0 wt % and hyaluronic acid or its pharmaceutically acceptable salt at 0.5-5.0 wt %, wherein the piroxicam or its pharmaceutically acceptable salt and hyaluronic acid or its pharmaceutically acceptable salt is contained at a weight ratio between 1:1 and 1:3.

The present invention is explained in details as follows.

The present invention provides a liquid pharmaceutical composition comprising piroxicam or its acceptable salt and hyaluronic acid or its acceptable salt at 0.25-10.0 wt % and 0.5-5.0 wt %, respectively, per total weight, and preferably 0.33-3.0 wt % and 1.0-3.0 wt %, wherein its weight ratio is between 1:1 and 1:3.

The liquid pharmaceutical composition in accordance with the present invention is preferably prepared as an injectable formulation, to be administered as an intra-articular injection to treat osteoarthritis.

In the present invention, pharmaceutically acceptable salts of piroxicam refer to all organic or inorganic addition salts of piroxicam, the concentration of which is relatively nontoxic and harmless to patients and the side effects of which do not degrade the beneficial effects of the piroxicam salt compound. Such a salt may use an inorganic acid or an organic acid as a free acid. Acceptable inorganic acids include hydrochloric acid, bromic acid, nitric acid, sulfuric acid, perchloric acid, and phosphoric acid. Such organic acids include citric acid, acetic acid, lactic acid, maleic acid, fumaric acid, gluconic acid, glyconic acid, succinic acid, tartaric acid, galacturonic acid, embonic acid, glutamic acid, aspartic acid, oxalic acid, D- or L-malic acid, methanesulfonic acid, ethanesulfonic acid, 4-toluenesulfonic acid, salicylic acid, benzoic acid, and malonic acid. Also, these salts can be alkali metal salts (e.g. sodium salts, potassium salts) as well as alkaline earth metal salts (e.g. calcium salts, magnesium salts). For example, acid addition salts can include acetate, aspartate, benzoate, besylate, bicarbonate/carbonate, bisulfate/sulfate, borate, camsylate, citrate, edisilate, ethylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthalate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, and zinc salt. Preferably, alkali metal salts, amines, and amino acid salts such as arginine and lysine may be used. A piroxicam salt, generated with one of these pharmaceutically acceptable salts, can be used to initiate the preparation of the aforementioned composition. Alternatively, a salt can be added dropwise midway in the preparation process, when piroxicam is being dissolved in a solvent. After sufficient stirring, piroxicam salt can be formed.

Furthermore, the present invention comprises hyaluronic acid, a pharmaceutically acceptable salt of hyaluronic acid, or a mixture of hyaluronic acid and a pharmaceutically acceptable salt thereof. Pharmaceutically acceptable salts of hyaluronic acid include inorganic salts such as sodium hyaluronate, magnesium hyaluronate, zinc hyaluronate, cobalt hyaluronate, as well as organic salts such as tetrabutylammonium hyaluronate. In some cases, a combination of two or more of these salts can be used. The molecular weight of hyaluronic acid in the present invention is not particularly limited, but an average molecular weight between 500,000 and 10,000,000 is preferred.

A liquid pharmaceutical composition according to the present invention comprises piroxicam and hyaluronic acid at 0.25-10.0 wt % and 0.5-5.0 wt %, respectively, and preferably at 0.33-3.0 wt % and 1.0-3.0 wt %, and its appropriate weight ratio is 1:1 to 1:3. No additive or synergistic treatment of osteoarthritis is expected above or below this ratio.

Piroxicam or its salt can make up 0.25-10.0 wt % of the total injection solution. Treatment effect is minimal at a concentration below 0.25 wt %. A concentration above 10.0 wt % requires excessive amount of solubilizers, which can lead to floating of the solubilizers or precipitation of piroxicam.

Moreover, the liquid composition may comprise hyaluronic acid or its salt at 0.5-5.0 wt % (by the concentration of hyaluronic acid) of the total solution. If the concentration of hyaluronic acid is at or below 0.5 wt %, treatment effect is insufficient. At a level above 5.0 wt %, the abrupt increase in the viscosity of the composition causes difficulty filling the pre-filled syringe or ampoule containers and difficulty administrating the composition to the affected part of the patient's body.

The preferable pH of the liquid composition of the liquid injectable formulation of hyaluronic acid and piroxicam is 5.5-8.5, and preferably 7.0-8.5. At a pH below 5, upon refrigeration or room temperature storage, the physical stability of piroxicam is decreased, possibly causing precipitation. At a pH level above 8.5, hyaluronic acid becomes unstable, and intra-articular administration can cause local irritation which can cause pain, edema, inflammation, and other side effects.

### Advantageous Effects of Invention

The present invention provides an intra-articular liquid injectable formulation, with synergistic effect on the treatment of osteoarthritis from the liquid composition comprising piroxicam or pharmaceutically acceptable salt thereof at 0.25-10.0 wt %, and hyaluronic acid or pharmaceutically acceptable salt thereof at 0.5-5.0 wt %, wherein the weight ratio of piroxicam or pharmaceutically acceptable salt thereof and hyaluronic acid or pharmaceutically acceptable salt thereof is between 1:1 and 1:3.

### BRIEF DESCRIPTION OF DRAWINGS

FIG. 1 shows reduction of inflammation in osteoarthritis-induced rat model, wherein the ratio of piroxicam and hyaluronic acid is (a) 1:4, (b) 1:3, (c) 1:2, (d) 1:1, (e) 2:1, and (f) 3:1.

FIG. 2 shows reduction of pain in osteoarthritis-induced rat model, wherein the ratio of piroxicam and hyaluronic acid is (a) 1:4, (b) 1:3, (c) 1:2, (d) 1:1, (e) 2:1, and (f) 3:1.

### BEST MODE FOR CARRYING OUT THE INVENTION

The present invention is explained in detail with examples to facilitate understanding. However, the examples according to the present invention can be modified into various forms and shall not be construed as limiting the scope of the present invention. The examples of the present invention are provided herein in order to describe the present invention thoroughly to the person of ordinary skill in the relevant art.

### Example 1

### Preparation of Liquid Composition Comprising Piroxicam and Hyaluronic Acid According to the Present Invention

Into approximately 350 ml of phosphate buffered saline (pH 7.4), 5.58 g of piroxicam potassium and an appropriate amount of solubilizing agent were added. Then, the mixture was stirred to dissolution for 1 hour at 30° C., and then buffered saline solution was added to a final volume of 500 ml. It was sterilized with a syringe filter. And then, 5.0 g of sodium hyaluronate was added. It was then stirred for 12 hours at 30-40° C. using an overhead mixer as a final step to preparing the composition.

### Examples 2 and 3

### Preparation of Liquid Compositions Comprising Piroxicam and Hyaluronic Acid According to the Present Invention

Liquid compositions comprising piroxicam and hyaluronic acid were prepared using the same method as Example 1 above but with contents as indicated in Table 1 below, and named Example 2 and Example 3, respectively.

### Comparative Examples 1-3

### Preparation of Liquid Compositions Comprising Piroxicam and Hyaluronic Acid According to the Present Invention

Liquid compositions comprising piroxicam and hyaluronic acid were prepared using the same method as Example 1 but with composition and contents as indicated in Table 1 below, and named Comparative Examples 1 through 3, respectively.

### Control Groups 1-7: Preparation of Liquid Compositions Comprising Piroxicam

Piroxicam liquid compositions were prepared using the same method as Example 1 but with composition and contents as indicated in Table 1 below, and named Control Groups 1 through 7, respectively.

TABLE 1

| | Piroxicam K | | | |
|---|---|---|---|---|
| | (piroxicam %) | HA | Pi:HA | pH |
| Example 1 | 1% | 1% | 1:1 | 7.4 |
| Example 2 | 0.5% | 1% | 1:2 | 7.4 |
| Example 3 | 0.33% | 1% | 1:3 | 7.4 |
| Comparative Example 1 | 0.25% | 1% | 1:4 | 7.4 |
| Comparative Example 2 | 2% | 1% | 2:1 | 7.4 |
| Comparative Example 3 | 3% | 1% | 3:1 | 7.4 |
| Control Group 1 | — | 1% | — | 7.4 |
| Control Group 2 | 0.25% | — | | 7.4 |
| Control Group 3 | 0.33% | — | | 7.4 |
| Control Group 4 | 0.5% | — | | 7.4 |
| Control Group 5 | 1% | — | | 7.4 |
| Control Group 6 | 2% | — | | 7.4 |
| Control Group 7 | 3% | | | 7.4 |

Pi: Piroxicam potassium
HA: Sodium hyaluronate

### Experiment 1: Assessment of Anti-Inflammatory Efficacy on Mono-Iodoacetate Induced Osteoarthritis Model

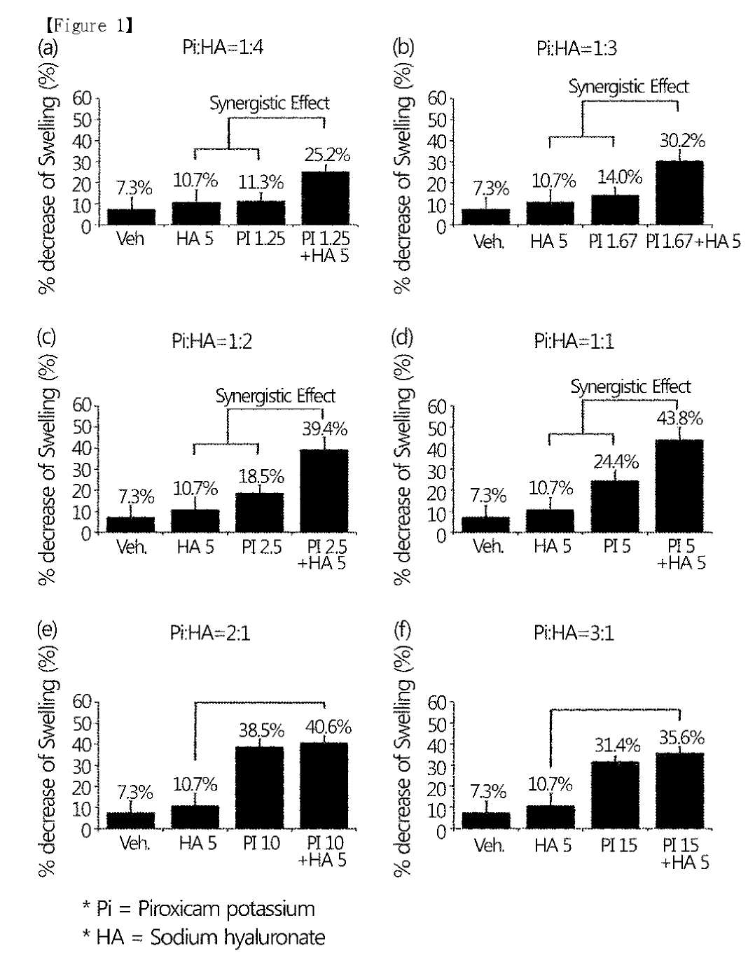

For this assessment, 8-week-old male SD rats from DBL Co., Ltd. were stabilized for four weeks in a cleanroom (laboratory animal room on the second basement floor of new wing of the research institute) where the temperature was maintained at 23±2° C., relative humidity at 40-60%, ventilation frequency at over 10 times/hour, ammonia concentration at or below 20 ppm, average illuminance at 150-300 lux, noise level at or below 60 phon, and light and dark cycle of 12 hours. At the beginning of the experiment, 3 mg/30 μl/head of mono-iodoacetate (hereinafter referred to as “MIA”) was administered to the right articular cavity. One day after administration, the rats were measured for the diameter of their joints, and they were grouped so that each group had the same average joint diameter. After forming the groups, the appropriate drug was administered once to each group. The diameters of joints were measured on day 3 of MIA administration. The results are illustrated in FIG. 1.

### Test Schedule:

Day 0: Joint diameter measured; Osteoarthritis induced
Day 1: Joint diameter measured; Drug administered
Day 3: Joint diameter measured

| Test | | | | |
|---|---|---|---|---|
| Group | Animal | Qty | Test substance | Ratio |
| G1 | SD rat | 7 | Vehicle i.a. | |
| G2 | SD rat + | 7 | Vehicle i.a. | |
| G3 | MIA 3 | 7 | HA 5 mg/head i.a. (Control Group 1) | |
| G4 | mg/head | 7 | Pi 1.25 mg/head i.a. (Control | |
| | | | Group 2) | |
| G5 | | 7 | Pi 1.67 mg/head i.a. (Control | |
| | | | Group 3) | |
| G6 | | 7 | Pi 2.5 mg/head i.a. (Control Group 4) | |
| G7 | | 7 | Pi 5 mg/head i.a. (Control Group 5) | |
| G8 | | 7 | Pi 10 mg/head i.a. (Control Group 6) | |
| G9 | | 7 | Pi 15 mg/head i.a. (Control Group 7) | |
| G10 | | 7 | Pi 1.25 + HA 5 mg/head i.a. | 1:4 |
| | | | (Comparative Example 1) | |
| G11 | | 7 | Pi 1.67 + HA 5 mg/head i.a. | 1:3 |
| | | | (Example 3) | |
| G12 | | 7 | Pi 2.5 + HA 5 mg/head i.a. | 1:2 |
| | | | (Example 2) | |
| G13 | | 7 | Pi 5 + HA 5 mg/head i.a. | 1:1 |
| | | | (Example 1) | |
| G14 | | 7 | Pi 10 + HA 5 mg/head i.a. | 2:1 |
| | | | (Comparative Example 2) | |
| G15 | | 7 | Pi 15 + HA 5 mg/head i.a. | 3:1 |
| | | | (Comparative Example 3) | |

### Test Groups:

Assessment of inflammation level: Edema caused by inflammatory changes in the joint was used as an indicator for the severity of inflammation. The joint diameter was used as the endpoint. Digimatic calipers (Mitutoyo, Japan) were used to measure the diameter of the joint.

Joint swelling=Absolute(Right joint diameter−Left joint diameter)

As seen in FIG. 1, there was a synergistic anti-inflammatory effect in Comparative Example 1 (Pi:HA=1:4), whereas Comparative Example 2 (Pi:HA=2:1) and Comparative Example 3 (Pi:HA=3:1) did not show synergistic effect on the anti-inflammatory action.

As shown, Example 1 (Pi:HA=1:1), Example 2 (Pi:HA=1:2), and Example 3 (Pi:HA=1:3) according to the present invention, all showed synergistic effect in inflammation reduction compared to control groups to which only piroxicam or only hyaluronic acid was administered.

### Experiment 2: Assessment of Analgesic Efficacy on Mono-Iodoacetate Induced Osteoarthritis Model

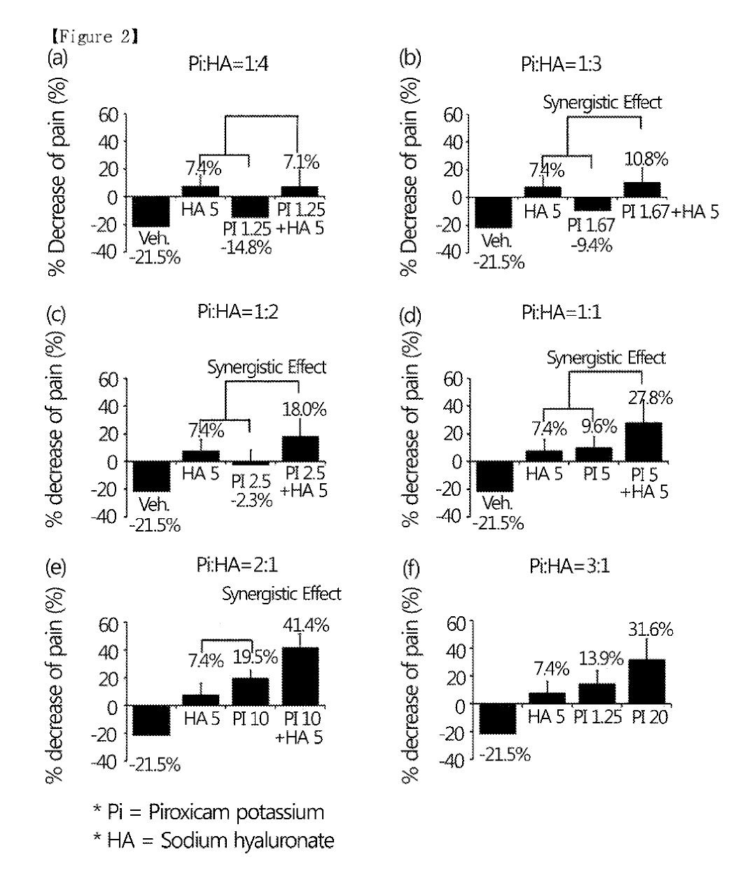

For this assessment, 8-week-old male SD rats from DBL Co., Ltd. were stabilized for four weeks in a cleanroom (laboratory animal room on the second basement floor of new wing of the research institute) which was maintained at a temperature of 23±2° C., relative humidity of 40-60%, ventilation frequency of over 10 times/hour, ammonia concentration at or below 20 ppm, average illuminance of 150-300 lux, noise level at or below 60 phon, and light and dark cycle of 12 hours. At the beginning of the experiment, 3 mg/30 μl/head of mono-iodoacetate (hereinafter referred to as “MIA”) was administered to the right joint space. One day after administration, the rats were measured for the weight balance and grouped so that each group had the same average. After forming the groups, appropriate drug was administered once to each group. Weight balance was measured on day 3 of MIA administration. The results are illustrated in FIG. 2.

### Test Schedule:

Day 0: Weight balance measured; Osteoarthritis induced
Day 1: Weight balance measured; Drug administered
Day 3: Weight balance measured

### Test Groups:

| Test | | | | |
|---|---|---|---|---|
| Group | Animal | Qty | Test substance | Ratio |
| G1 | SD rat | 7 | Vehicle i.a. | |
| G2 | SD rat + | 7 | Vehicle i.a. | |
| G3 | MIA 3 | 7 | HA 5 mg/head i.a. (Control Group 1) | |
| G4 | mg/head | 7 | Pi 1.25 mg/head i.a. (Control | |
| | | | Group 2) | |
| G5 | | 7 | Pi 1.67 mg/head i.a. (Control | |
| | | | Group 3) | |
| G6 | | 7 | Pi 2.5 mg/head i.a. (Control Group 4) | |
| G7 | | 7 | Pi 5 mg/head i.a. (Control Group 5) | |
| G8 | | 7 | Pi 10 mg/head i.a. (Control Group 6) | |
| G9 | | 7 | Pi 15 mg/head i.a. (Control Group 7) | |
| G10 | | 7 | Pi 1.25 + HA 5 mg/head i.a. | 1:4 |
| | | | (Comparative Example 1) | |
| G11 | | 7 | Pi 1.67 + HA 5 mg/head i.a. | 1:3 |
| | | | (Example 3) | |
| G12 | | 7 | Pi 2.5 + HA 5 mg/head i.a. | 1:2 |
| | | | (Example 2) | |
| G13 | | 7 | Pi 5 + HA 5 mg/head i.a. | 1:1 |
| | | | (Example 1) | |
| G14 | | 7 | Pi 10 + HA 5 mg/head i.a. | 2:1 |
| | | | (Comparative Example 2) | |
| G15 | | 7 | Pi 15 + HA 5 mg/head i.a. | 3:1 |
| | | | (Comparative Example 3) | |

Assessment of Pain Level:

Weight distribution (%), which is the most commonly used method of assessing pain levels, was used. In calculating the weight distribution, the rat was placed on an incapacitance meter, and when both hind leg soles were on the measuring board, the average weight measured for 5 seconds was obtained of each hind leg, using the following formula:

Weight distribution (%)=100*right limb weight/(left limb weight+right limb weight)

All results are indicated in means±SEM. To determine the significance of the experiment results, Student's t-test of SigmaStat was used for statistical processing of each group.

As shown in FIG. 2, only Comparative Example 2 (Pi:HA=2:1) showed synergistic effect in pain reduction, whereas Comparative Example 1 (Pi:HA=1:4) and Comparative Example 3 (Pi:HA=1:3) did not show synergistic effect in pain reduction.

Example 1 (Pi:HA=1:1), Example 2 (Pi:HA=1:2), and Example 3 (Pi:HA=1:3) according to this invention showed synergistic effect in pain reduction compared to control groups to which only piroxicam or only hyaluronic acid was administered.

As seen above, the present invention has synergistic anti-inflammatory and analgesic effects compared to control groups. Therefore, the present invention is very useful as an anti-inflammatory analgesic pharmaceutical composition.

1. A pharmaceutical composition for the treatment of osteoarthritis comprising piroxicam or pharmaceutically acceptable salt thereof and hyaluronic acid or pharmaceutically acceptable salt thereof,
wherein the composition comprises piroxicam or pharmaceutically acceptable salt thereof at 0.25-10.0 wt % and hyaluronic acid or pharmaceutically acceptable salt thereof at 0.5-5.0 wt %, and the weight ratio of piroxicam or its pharmaceutically acceptable salt thereof and hyaluronic acid or its pharmaceutically acceptable salt thereof is between 1:1 and 1:3.

2. The composition according to claim 1, wherein the concentration of piroxicam or pharmaceutically acceptable salt thereof is 0.33 wt %-3.0 wt % and the concentration of hyaluronic acid or pharmaceutically acceptable salt thereof is 1.0 wt %-3.0 wt %.

3. The composition according to claim 1, wherein the pH of the said pharmaceutical composition is between 5.5 and 8.5.

4. The composition according to claim 3, wherein the pH of the composition is between 7.0 and 8.5.

5. The composition according to claim 1, wherein the pharmaceutically acceptable salt of piroxicam is selected from the group consisting of hydrochloric acid, bromic acid, nitric acid, sulfuric acid, perchloric acid, phosphoric acid, citric acid, acetic acid, lactic acid, maleic acid, fumaric acid, gluconic acid, glycolic acid, succinic acid, tartaric acid, galacturonic acid, embonic acid, glutamic acid, aspartic acid, oxalic acid, D- or L-malic acid, methanesulfonic acid, ethanesulfonic acid, 4-toluenesulfonic acid, salicylic acid, benzoic acid, malonic acid, sodium salt and potassium salt, which are alkali metals, and calcium salt and magnesium salt, which are alkaline earth metal salts.

6. The composition according to claim 5, wherein the pharmaceutically acceptable salt of piroxicam is potassium salt.

7. The composition according to claim 1, wherein the pharmaceutically acceptable salt of hyaluronic acid is selected from the group consisting of sodium hyaluronate, magnesium hyaluronate, zinc hyaluronate, cobalt hyaluronate, and tetrabutylammonium hyaluronate.

8. The composition according to claim 7, wherein the pharmaceutically acceptable salt of hyaluronic acid is sodium salt.

9. The composition according to claim 7, wherein the pharmaceutically acceptable salt of hyaluronic acid is a mixture of two salts.

10. The composition according to claim 1, wherein the hyaluronic acid is a mixture of hyaluronic acid and pharmaceutically acceptable salt thereof.

11. The composition according to claim 1, wherein the pharmaceutical composition is prepared in the form of injectable formulation.

12. The composition according to claim 11, wherein the pharmaceutical composition is administered into articular cavity.
